(19) United States
(12) Patent Application Publication
(10) Pub. No.: US 2006/0058629 A1
(43) Pub. Date: Mar. 16, 2006

(54) Magnetic resonance method

(76) Inventors: Jan Bertus Marten Warntjes, Eindhoven (NL); Johan Samuel Van Den Brink, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/536,285

(22) PCT Filed: Nov. 20, 2003
(86) PCT No.: PCT/IB03/05290

§ 371 Date: May 25, 2005

(30) Foreign Application Priority Data
Nov. 26, 2002 (EP) 02079908.6

(51) Int. Cl. A61B 5/05

(52) U.S. Cl. 600/410

(57) ABSTRACT

A novel magnetic resonance imaging method is presented for forming an image of an object from a plurality of signals acquired by an array of multiple receiver antennae. Prior to imaging a sensitivity map of each of the receiver antennae is provided, at least two adjacent antennae record signals originating from the same imaging position and the image intensity is calculated from the signals measured by different antennae, wherein the number of phase encoding steps is reduced with respect to the full set thereof. In addition the field of view is set smaller than the object size in phase encoding direction inducing intrinsic foldover artefacts, whereas the sensitivity map of the receiver antennae and a reference image featuring intrinsic foldover artefacts are used for reconstruction of the MR image to an unfolded image.

The invention relates to a magnetic resonance (MR) method for the imaging of an object arranged in a steady magnetic field, whereas the following steps being repeatedly executed according to said method:
excitation of spins in a part of the object,
measurement of MR signals along a predetermined trajectory containing a plurality of lines in k-space by application of a read gradient and other gradients,
application of a navigator gradient for the measurement of navigator MR-signals,
said method also including the determination of a phase correction from phases and moduli of the measured navigator MR signals so as to correct the measured MR signals and the determination of an image of the part of the object from the corrected MR signals.

The invention also relates to an MR device and a computer program product for carrying out such a method.

In U.S. Pat. No. 2002/0013526 an inherently de-coupled sandwiched solenoidal array coil is described for use in receiving NMR radio frequency signals in both horizontal and vertical field MRI systems. In its most basic configuration, the array coil comprises two coaxial RF receiver coils. The first coils of the array has two solenoidal (or loop) sections that are separated form one another along a common axis. The two sections are electrically connected in series but the conductors in each section are wound in opposite directions so that a current through the coil sets up a magnetic field of opposite polarity in each section. The second coil of the coil array is disposed (“sandwiched”) between the two separated solenoidal sections of the first coil in a region where the combined opposing magnetic fields cancel to become a null. Due to the winding arrangement and geometrical symmetry, the receiver coils of the array become electromagnetically “de-coupled” from one another while still maintaining their sensitivity toward receiving NMR signals. The multiple coil array arrangement also allows for selecting between a larger or smaller filed-of-view (FOV) to avoid image fold-over problems without time penalty in image data acquisition. Also alternative embodiments are disclosed which include unequal constituent coil diameters, unequal constituent coil windings, non-coaxial coil configurations etc.

With the coil array arrangement of this reference the FOV can be chosen to be large by combining the NMR signals from several coils of the array or to be small by selecting only the NMR signals of a single coil, in order to overcome fold-over artefacts if an image is obtained from a small region or volume of interest. Thus, the FOV can be selected dependent from the size of the imaging object.

Further, in EP-A-1 102 076 a magnetic resonance imaging method is disclosed, in which magnetic gradient fields in a phase-encode and read-out direction are applied for spatially encoding excited MR active nuclei in a region of interest of a patient. A reduced number of readings in the read-out direction is taken, thereby creating an aliased reduced field of view image. At least two RF receive coils are used together with sensitivity information concerning those coils in order to unfold the aliased image to produce a full image while taking advantage of the reduced time of collection of data. The sensitivity information is collected at a lower resolution than that at which the image information is collected. The effect of lower resolution in the reference data, used to calibrate the sensitivity of the coils, is to reduce noise in the reference data and thus the signal-to-noise of the target unfolded SENSE data is increased.

If the FOV is smaller than the imaged object intrinsic foldover artefacts will occur. Intrinsic foldover artefacts are used in e.g. cardiac imaging, where the region of interest, the heart, is much smaller than the object slice, or in imaging the abdomen, where the arms are fold-in, and in whole body MR imaging, where the deformed edges of the large FOV are not used. In a parallel imaging method like SENSE or SMASH it is not allowed to choose a field of view that is smaller than the object size in the phase encoding direction, as intrinsic foldover artefacts make the coil sensitivity matrices undetermined. If SENSE is used, the operator is forced to choose a large field-of-view encompassing the whole object, which partly wastes the time reduction provided by the SENSE method.

It is now an object of the present invention to further enhance the efficiency of parallel imaging techniques as SENSE or SMASH.

This object of the invention are achieved by a method as defined in claim 1.

The invention is further related to an apparatus as defined in claim 4 and to a computer program product as defined in claim 5.

The present invention has the main advantage that a reduced FOV can be chosen. As a consequence that intrinsic foldover artefacts are generated which however can be resolved by calculation of the reference image.

These and other advantages of the invention are disclosed in the dependent claims and in the following description in which an exemplified embodiment of the invention is described with respect to the accompanying drawings. Therein shows:

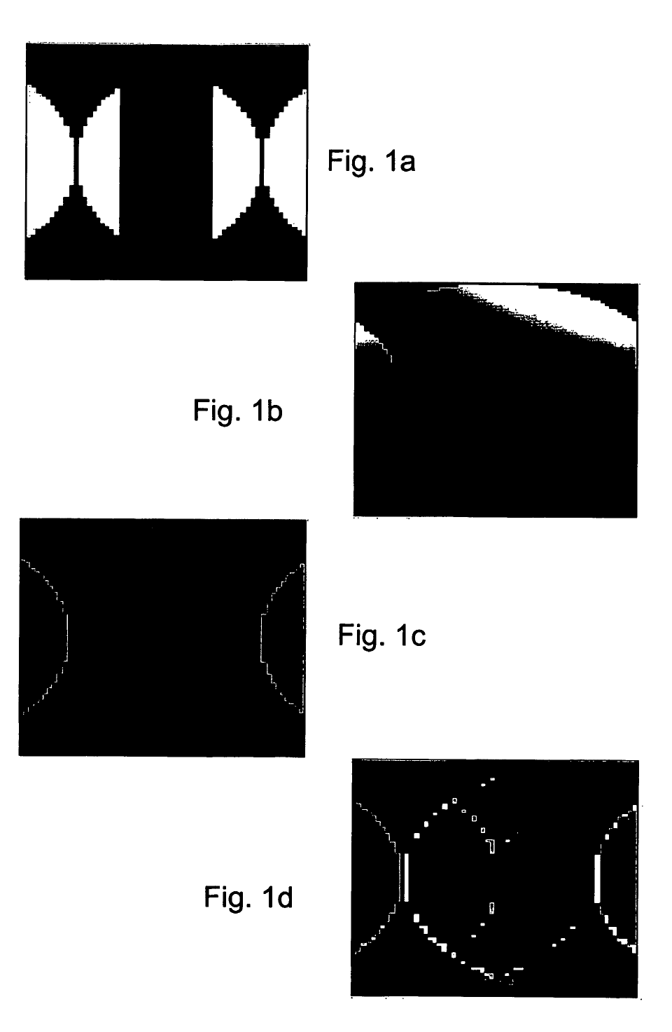

FIG. 1 a SENSE reconstruction with a small FOV showing artefacts,

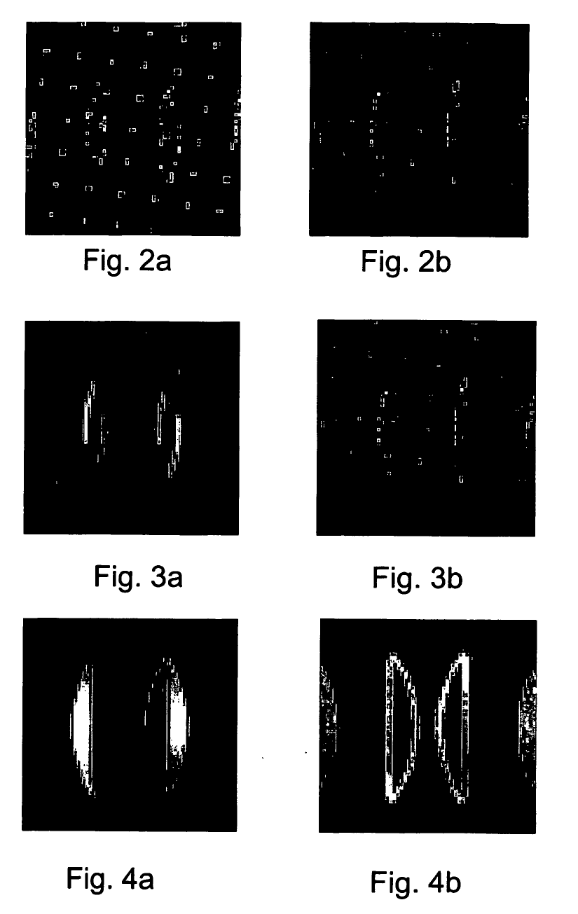

FIG. 2 a SENSE reconstructed MR image from a phantom with equidistant columns of water,

FIG. 3 a SENSE reconstructed MR image from a phantom as in FIG. 2 with additionally large water columns on its sides,

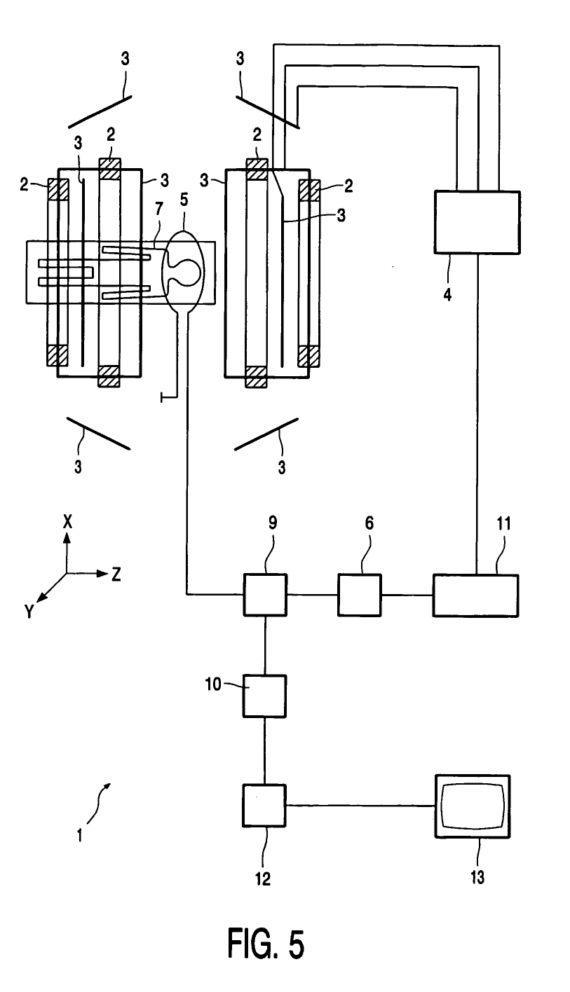

FIG. 4 a SENSE reconstructed MR from a homogeneously filled water phantom, and

FIG. 5 an apparatus for carrying out the method in accordance with the present invention.

In magnetic resonance imaging there is a general tendency to obtain acceptable images within shorter periods of time. For this reason the sensitivity encoding method called “SENSE” has recently been developed by the Institute of Biomedical Engineering and Medical Informations, University and ETH Zurich, Switzerland. The SENSE method is based on an algorithm which acts directly on the image as detected by the multiple coils of the magnetic resonance apparatus. The number of phase encoding steps for an image is reduced by a factor R leading to a acceleration of the signal acquisition by that factor, where R can be any number larger than 1. That is, the number of (phase) encoding steps is reduced with respect to a full set of encoding steps. This full set induces the encoding steps required for sampling MR-signals in k-space sufficient for a pre-selected spatial resolution of the MR-image that is reconstructed. The resulting aliased images from the multiple coils are used by the SENSE algorithm to generate a single, R times unfolded image. Crucial for the SENSE method is the knowledge of the sensitivity of the coils which are arranged in so called sensitivity maps. In order to accelerate this method there are proposals to use raw sensitivity maps which can be obtained through division by either the “sum-of-squares” of the single coil references or by an optional body coil reference (see e.g. K. Pruessmann et. al. in Proc. ISMRM, 1998, abstracts pp. 579, 799, 803 and 2087). In fact the SENSE method allows for a decrease in scan time by deliberately undersampling k-space, i.e. deliberately selecting a Field of view (FOV) that is smaller than the object to be acquired. This undersampling causes fold-over artefacts which can be resolved or unfolded by the use of the knowledge of a set of distinct coils having different coil sensitivity patterns. The undersampling can be in either one of both phase-encoding directions.

In NMR imaging the method of intrinsic foldover artefacts is used e.g. in cardiac imaging, where the region of the interest, i.e. the heart, is much smaller than the object slice, or for imaging abdomen, where the arms are fold in, or in whole body scans, where the deformed edges of the large FOV are not used. In a parallel imaging method like SENSE it is normally not allowed to choose a field of view that is smaller than the object size in the phase encoding direction, since the intrinsic foldover artefacts make the coil sensitivity matrices undetermined. Using the SENSE method the operator is forced to choose a large field of view encompassing the whole object, which partly wastes time reduction provided by SENSE. This restriction is believed to be impossible to overcome on the basis of the mathematics used in parallel imaging methods as SENSE.

At the moment a SENSE measurement is basically provided as follows:
1. A prescan is made to obtain low-resolution images for each element of the coils used in the SENSE method, without the anatomical details of the patient.
2. The SENSE scan is performed resulting in aliased images of all elements.
3. The sensitivity profiles of the coils and the SENSE scan images are used by the SENSE algorithm to reconstruct the actual image. The body coil reference scan is also used for regularization. Normally one prescan is sufficient for all SENSE scans in a particular MR-examination.

In order to allow intrinsic foldover artefacts one extra step should be added after step 2:

2b. From the images of the prescan the sensitivity profiles of the coil elements featuring intrinsic foldover and the reference image featuring intrinsic foldover are calculated. This can be done within a fraction of a second, as part of the reconstruction process or even during the scan. Subsequently these explicitly folded images are used in the SENSE algorithm.

With this method one can gain a time reduction of about 30%. As an example an object of 40 cm width is taken. For a 1 mm resolution 400 encoding steps are needed. If the region of interest is only 20 cm and intrinsic foldover artefacts are allowed, a field of view of 30 cm can be chosen and 300 encoding steps are necessary. If one uses only SENSE with a reduction factor of 3 and necessarily a field of view of 40 cm, only 133 encoding steps are needed. If in addition intrinsic foldover artefacts with the SENSE method are allowed and a field of view of 30 cm is used, only 100 encoding steps are needed. The noise in the region of interest is equal to the noise of the image made by the normal SENSE method with a reduction factor of 3. However, the presented method with intrinsic foldover artefacts is 30% faster.

At the time of introduction of SENSE is was believed that any combination with intrinsic foldover artefacts was impossible, based on the mathematical principles of the SENSE method. However, the experiment shows that in practice the above mentioned method works very well, although mathematics of SENSE still holds. Consider following example: the field of view FOV is defined as being ¾*object, (mFOV=0.75 & object), and a SENSE reduction factor of 3 is applied with the reduced field of view mFOV, i.e. the SENSE foldover distance Δx=⅓*mFOV=¼*object. Now, a pixel m in the aliased images of all coil elements i derive signals from 4 positions in the object, owing to the SENSE reduction factor of 3 plus the intrinsic foldover artefacts:
mᵢ=cᵢ(x)·S(x)+cᵢ(x+Δx)·S(x+Δx)+cᵢ(x+2Δx)·S(x+2Δx)+c(x+3Δx)·S(x+3Δx) eq. (1)
where cᵢ(x) is the sensitivity of coil element i at position x and S(x) is the RF signal received from position x. Since only the signal intensities S(x+Δx) and S(x+2Δx) are within the region of interest and can be reconstructed without any corruption, the factual values of the signals S(x) and S(x+3Δx) are not important anymore. If a SENSE factor of 3 is applied on the reduced mFOV, for pixel mᵢ can be written:
mᵢ=c_{i,eff}(x)·S_{eff}(x)+cᵢ(x+Δx)·S(x+Δx)+cᵢ(x+2Δx)·S(x+2Δx) eq. (2)
The factor c_{i,eff}(x) can be obtained by acquiring the reference data at a reduced mFOV before each scan, which method is slow and undesirable, or c_{i,eff}(x) can be approximated by applying an explicit folding of the reference data at reconstruction and assuming that:
c_{i,eff}(x)=cᵢ(x)+cᵢ(x+3Δx) eq. (3)
That is effective antennae sensitivities are denied from antennae sensitivity value that are an integer multiple of the reduced FOV (Δx) apart. The only concern in a mathematical point of view lies in this approximation since c_{i,eff} being a division of the complex coil element signal Sᵢ by the complex quadrature body coil signal S_{QBC} and thus

$${C}_{i} {,}_{eff} ( x ) = \\ {S}_{i} ( x ) + \frac{{S}_{i} ( x + 3 Δ x )}{{S}_{QBC} ( x )} + {S}_{QBXC} ( x + 3 Δ x ) \neq \frac{{S}_{i} ( x )}{{S}_{QBC} ( x )} + \frac{{S}_{i} ( x + 3 Δ x )}{{S}_{QBC} ( x + 3 Δ x )} = {C}_{i} ( x ) + {C}_{i} ( x + 3 Δ x )$$

However, since the sensitivity of a coil element drops off fast with distance, one of the two elements in equation (4) is always much larger than the other and the approximation will be valid.

For the simulation of focussed SENSE as described above an MRI device with an 8 element headcoil was used to obtain 8 sensitivity maps. The resolution of the sensitivity maps is equal to the resolution of the SENSE image. As reference a 16 cm diameter water filled phantom and a FOV of 14×16 cm² was used so that there is an intrinsic foldover artefact in the image. The sensitivity maps have been measured over a larger volume. A SENSE reconstruction with a small mFOV will show artefacts as can be seen in FIG. 1a. When the sensitivity maps are artificially backfolded as in step 2b above. The resulting (modulus) image of one element in the 14×16 cm² FOV is displayed in FIG. 1b. The element is positioned on the top right side. If the backfolded sensitivity maps are used as input, the SENSE reconstruction will work fine. After reconstruction only the intrinsic foldover artefacts are left as shown in FIG. 1c. Normally the resolution of the sensitivity maps is chosen smaller than the resolution of the actual SENSE image. As shown in FIG. 1d a backfolded sharp edge can lead to these artefacts. In a lot of cases, however, the sensitivity at the backfolded edge is low (e.g. in cardiac images) and the artefact is by far not so pronounced and thus can be neglected.

FIGS. 2 to 4 show the images in which a phantom is measured with a SENSE factor of 3 in the Left to Right (LR) direction. The field of view is chosen smaller than the phantom, leading to intrinsic fold-over artefacts. Upon unfolding with SENSE the sensitivity estimation is wrong due to this intrinsic fold-over which leads to severe artefacts as can be seen in the left set of images. In FIG. 2a a phantom with equidistant columns of water is used, in FIG. 3a the same phantom as in FIG. 2a with additionally large columns on the sides of the phantoms, and in FIG. 4a a homogeneously filled water phantom. These images show what normally happens if the operator chooses a too small FOV. The right set images of the same phantoms are taken with SENSE including the intrinsic foldover algorithm. The intrinsic foldover artefacts disappear except for the intrinsic foldover of the edges of the object. In this quick feasibility example the reference scan was taken with a lower resolution than the actual SENSE scan which leads to the ringing artefacts as also shown in the simulation of FIG. 1d. A better match of the resolution of both scans and the application of a ringing filter will improve the image. Note that in this example the overall sensitivity at the edges is much larger than the overall sensitivity in the center of the object in this particular coil, leading to a strong edge artefact. For the coil it was intended, the SENSE cardiac coil, the edge artefacts will be greatly reduced if the phase encode direction is chosen anywhere in the coronal plane because the sensitivity on the edges in a coronal plane is lower than in the center. Thus the images in FIGS. 2b, 3b and 4b are unfolded correctly, showing a clear region of interest and remaining some foldover artefacts on the sides.

A practical embodiment of an MR device is shown in FIG. 5, which includes a first magnet system 2 for generating a steady magnetic field, and also means for generating additional magnetic fields having a gradient in the X, Y, Z directions, which means are known as gradient coils 3. However, since the coils 3 are highly non-linear as mentioned above, the field patterns or “gradients” are not directed only in one of the X, Y and Z directions as in usual MR systems. The Z direction of the co-ordinate system shown corresponds to the direction of the steady magnetic field in the magnet system 2 by convention, which only should be linear. The measuring co-ordinate system x, y, z to be used can be chosen independently of the X, Y, Z system shown in FIG. 2. The gradient coils or antennae are fed by a power supply unit 4. An RF transmitter coil 5 serves to generate RF magnetic fields and is connected to an RF transmitter and modulator 6. A receiver coil is used to receive the magnetic resonance signal generated by the RF field in the object 7 to be examined, for example a human or animal body. This coil may be the same coil as the RF transmitter coil 5 or an array of multiple receiver antennae (not shown). The coil 5 is a non phased-array receiver antenna, which is different from the array of multiple receiver antennae. Furthermore, the magnet system 2 encloses an examination space which is large enough to accommodate a part of the body 7 to be examined. The RF coil 5 is arranged around or on the part of the body 7 to be examined in this examination space. The RF transmitter coil 5 is connected to a signal amplifier and demodulation unit 10 via a transmission/reception circuit 9. The control unit 11 controls the RF transmitter and modulator 6 and the power supply unit 4 so as to generate special pulse sequences which contain RF pulses and gradients. The control unit 11 also controls detection of the MR signal(s), whose phase and amplitude obtained from the demodulation unit 10 are applied to a processing unit 12. The control unit 11 and the respective receiver coils 3 and 5 are equipped with control means to enable switching between their detection pathways on a sub-repetition time basis (i.e. typically less than 10 ms). These means comprise inter alia a current/voltage stabilisation unit to ensure reliable phase behaviour of the antennae, and one or more switches and analogue-to-digital converters in the signal path between coil and processing unit 12. The processing unit 12 processes the presented signal values so as to form an image by transformation. This image can be visualized, for example by means of a monitor 13.

1. A magnetic resonance imaging method for forming an image of an object from a plurality of signals acquired by receiver antennae, a sensitivity map of each of the receiver antennae being provided, wherein the number of encoding steps applied is reduced with respect to the full set thereof, wherein the field of view is set smaller than the object size in an encoding direction and wherein the sensitivity map of the receiver antennae and a reference image featuring intrinsic foldover artefacts are used for reconstruction of the MR image into an unfolded image.

2. A method as claimed in claim 1, wherein an edge-filtering which removes edge-artefacts is applied to the unfolded image.

3. A method as claimed in claim 2, wherein a ringing-filter which removes ring-artefacts is applied to the unfolded image.

4. A magnetic resonance imaging apparatus for obtaining an MR image from a plurality of signals comprising:
means for excitation of spins in a part of the object,
a plurality of receiver antennae,
means for measuring MR signals along a predetermined trajectory containing a plurality of lines in k-space by application of a read gradient and other gradients, wherein the number of phase encoding steps is reduced with respect to the full set thereof,
means for providing a sensitivity map for each of the receiver antennae,
means for setting the field of view smaller than the object size, and
means for reconstructing the MR image to an unfolded image from the measured MR signals by using the sensitivity maps of the receiver antennae and a reference image featuring intrinsic foldover artefacts.

5. A computer program product stored on a computer usable medium for forming an image by means of the magnetic resonance method, comprising a computer readable program means for causing the computer to control the execution of:
a magnetic resonance imaging apparatus for obtaining an MR image from a plurality of signals, the computer program comprising instructions for
setting the field of view smaller than the object size, and
reconstructing the MR image to an unfolded image from measured MR signals by using sensitivity maps of the receiver antennae and a reference image featuring intrinsic foldover artefacts.
